(19) United States
(12) Patent Application Publication
(10) Pub. No.: US 2010/0256179 A1
(43) Pub. Date: Oct. 7, 2010

(54) COMBINATION THERAPY FOR PAIN IN PAINFUL DIABETIC NEUROPATHY

(75) Inventors: Thomas Stöhr, Monheim (DE); Bettina Beyreuther, Dusseldorf (DE)

Correspondence Address: HARNESS, DICKEY, & PIERCE, P.L.C, 7700 Bonhomme, Suite 400, ST. LOUIS, MO, 63105, US

(73) Assignee: UCB PHARMA GMBH, Monheim (DE)

(21) Appl. No.: 12/752,429

(22) Filed: Apr. 1, 2010

Related U.S. Application Data
(63) Continuation of application No. 11/506,577, filed on Aug. 18, 2006, now abandoned.

(30) Foreign Application Priority Data
Mar. 26, 2004 (EP) 04 007 360.3

(51) Int. Cl. A61K 31/485; A61P 25/02; A61K 31/197; A61K 31/381; A61K 31/165; A61P 31/00

(52) U.S. Cl. 514/289; 514/561; 514/438; 514/626

(57) ABSTRACT

A method for treating pain in painful diabetic neuropathy comprises administering in combination a first agent that comprises a compound as defined herein, illustratively lacosamide, and a second agent effective to provide enhanced treatment of pain, by comparison with the first agent alone. The second agent illustratively comprises an analgesic, an anticonvulsant, an antidepressant or an NMDA receptor antagonist.

This application is a continuation of U.S. application Ser. No. 11/506,577 filed Aug. 18, 2006. This application contains subject matter that is related to co-assigned U.S. application Ser. No. 11/089,441 filed on Mar. 25, 2005, which claims priority from U.S. provisional application Ser. No. 60/556,499 filed on Mar. 26, 2004 and European application No. EP 04 007 360.3 filed on Mar. 26, 2004. This application contains subject matter that is related to co-assigned U.S. application Ser. No. 11/506,523 titled “Method for treating non-inflammatory musculoskeletal pain”; to co-assigned U.S. application Ser. No. 11/506,578 titled “Method for treating non-inflammatory osteoarthritic pain”; and to co-assigned U.S. application Ser. No. 11/506,524 titled “Therapeutic combination for painful medical conditions”. The disclosure of each of the applications identified in this paragraph is incorporated herein by reference in its entirety.

Above-referenced U.S. application Ser. No. 11/089,441 published as U.S. Patent Application Publication No. 2006/0100157 on May 11, 2006, and a counterpart PCT application published as International Patent Publication No. WO 2005/092313 on Oct. 6, 2005. These publications are not admitted to be prior art to the present invention.

### FIELD OF THE INVENTION

The present invention relates to therapeutic methods for treating pain in painful diabetic neuropathy.

### BACKGROUND OF THE INVENTION

Diabetic neuropathies are a family of nerve disorders caused by diabetes which can be very painful. There are several causes of human neuropathy with considerable variability in symptoms and neurological deficits. Painful neuropathies are defined as neurological disorders characterized by persistence of pain and hypersensitivity in a body region of which the sensory innervation has been damaged, but damage to sensory nerves does not always produce neuropathic pain. Usually loss of sensation is observed rather than hypersensitivity or pain.

Pain is a subjective experience and the perception of pain is performed in particular parts of the central nervous system (CNS). Usually noxious (peripheral) stimuli are transmitted to the CNS beforehand, but pain is not always associated with nociception. A broad variety of different types of clinical pain exists, derived from different underlying pathophysiological mechanisms and needing different treatment approaches.

Three major types of clinical pain have been characterized: acute pain, chronic pain, and neuropathic pain.

Acute clinical pain may result, for example, from inflammation or soft tissue injury. This type of pain is adaptive and has the biologically relevant function of warning and enabling healing and repair of an already damaged body part to occur undisturbed. A protective function is achieved by making the injured or inflamed area and surrounding tissue hypersensitive to all stimuli so that contact with any external stimulus can be avoided. The neuronal mechanisms underlying this type of clinical pain are fairly well understood and pharmacological control of acute clinical pain is available and effective, for example by means of nonsteroidal anti-inflammatory drugs (NSAIDs) up to opioids depending on type and extent of the sensation of pain.

Chronic clinical pain appears as sustained sensory abnormalities resulting from an ongoing peripheral pathology such as cancer or chronic inflammation (e.g., arthritis) or it can be independent of such initiating triggers. Chronic pain that is independent of initiating triggers is maladaptive, offering no survival advantage, and very often no effective treatment is available.

Neuropathic pain can be classified as peripheral or central. Peripheral neuropathic pain is caused by injury or infection of peripheral sensory nerves, whereas central neuropathic pain is caused by damage to the CNS or/and the spinal cord. Both peripheral and central neuropathic pain can occur without obvious initial nerve damage.

The clinical causes of neuropathic pain are widespread and include both trauma and disease. Different neuropathic syndromes (for example diabetic neuropathy, postherpetic neuralgia, trigeminal neuralgia, postoperative pain, post-traumatic pain, HIV pain, cancer pain, etc.) have different underlying mechanisms.

Common analgesics, e.g., opioids and NSAIDs, insufficiently address chronic abnormal pain syndromes such as peripheral and central neuropathic pain due to insufficient efficacy or limiting side effects, although a subset of patients with neuropathic pain responds to opioids. In the search for alternative treatment regimes to produce satisfactory and sustained pain relief, corticosteroids, conduction blockade, glycerol, antidepressants, local anesthetics, gangliosides and electrostimulation have been tried. Anticonvulsants have been found useful against various types of peripheral neuropathic pain conditions. For example, gabapentin or pregabalin can be effective in reducing pain in patients with diabetic neuropathy. However, pregabalin, for instance, induces weight gain in Type I or II diabetes patients by edema formation. Increased weight is an established risk factor for cardiovascular disease, particularly in Type II diabetic patients.

Pain derived from a diabetic sensory neuropathy is the most common form of diabetic neuropathy. It is usually of insidious onset. Predominant pain may be combined with temperature and tactile loss. The pain is usually aching, prickling, or burning in quality with superimposed stabs, and often most troublesome at night. The pain is felt predominantly in the lower limbs, however, with occurrence also at the upper limbs and trunk.

If general overactivity and unleaded low threshold activation of sensory neurons is considered as one of the main syndromes of neuropathy and neuropathic pain sensation with a marked mechanoallodynia as the most disabling clinical symptom, selective inhibition of this pathophysiological event instead of general inhibition of high threshold noxious stimuli (e.g., by local anesthetics) of the normal sensory nociception provides clear advantages.

Certain peptides are known to exhibit CNS activity and are useful in treatment of epilepsy and other CNS disorders. Such peptides are described, for example, in U.S. Pat. No. 5,378,729.

Related peptides are disclosed in U.S. Pat. No. 5,773,475 as useful for treating CNS disorders.

International Patent Publication No. WO 02/074784, incorporated herein by reference in its entirety, relates to use of such peptide compounds having antinociceptive properties, for treatment of different types and symptoms of acute and chronic pain, especially non-neuropathic inflammatory pain, e.g., rheumatoid arthritic pain or secondary inflammatory osteoarthritic pain.

International Patent Publication No. WO 02/074297 relates to treatment of allodynia related to peripheral neuropathic pain, using a compound of formula

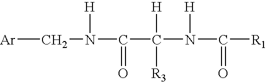

where Ar is a phenyl group that is unsubstituted or substituted with at least one halo substituent; R₃ is C₁₋₃ alkoxy; and R₁ is methyl.

Lacosamide (also called SPM 927 or harkoseride) is a compound of the above formula that has a mode of action which is so far unknown (Bialer et al. (2002) Epilepsy Res. 51:31-71). The mode of action of lacosamide and other peptide compounds disclosed in the above-referenced patents and publications differs from that of common antiepileptic drugs. Ion channels are not affected by these compounds in a manner comparable to other known antiepileptic drugs. For example, gamma-aminobutyric acid (GABA) induced currents are potentiated, but no direct interaction with any known GABA receptor subtype has been observed. Glutamate induced currents are attenuated but the compounds do not directly interact with any known glutamate receptor subtype.

The mechanisms of neuropathic pain in diabetic patients are poorly understood. Current treatments use a variety of pharmacological, surgical, physical and psychological approaches. However, the evidence for many of the treatments is still limited. Therefore, a need remains for improved therapies to treat pain in painful diabetic neuropathy.

### SUMMARY OF THE INVENTION

There is now provided a method for treating pain in painful diabetic neuropathy in a subject, comprising administering in combination to the subject a first agent that comprises a compound of Formula (I)

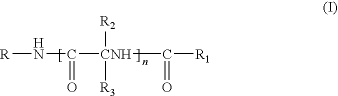

wherein:
R is hydrogen, lower alkyl, lower alkenyl, lower alkynyl, aryl, aryl lower alkyl, heterocyclic, heterocyclic lower alkyl, lower alkyl heterocyclic, lower cycloalkyl or lower cycloalkyl lower alkyl, and R is unsubstituted or is substituted with at least one electron withdrawing group, and/or at least one electron donating group;
R₁ is hydrogen or lower alkyl, lower alkenyl, lower alkynyl, aryl lower alkyl, aryl, heterocyclic lower alkyl, lower alkyl heterocyclic, heterocyclic, lower cycloalkyl, or lower cycloalkyl lower alkyl, and is unsubstituted or substituted with at least one electron-withdrawing group and/or at least one electron-donating group;
R₂ and R₃ are independently hydrogen, lower alkyl, lower alkenyl, lower alkynyl, aryl lower alkyl, aryl, halo, heterocyclic, heterocyclic lower alkyl, lower alkyl heterocyclic, lower cycloalkyl, lower cycloalkyl lower alkyl, or Z—Y, wherein R₂ and R₃ are each independently unsubstituted or substituted with at least one electron-withdrawing group and/or at least one electron-donating group;
Z is O, S, S(O)ₐ, NR₄, NR′₆, PR₄ or a chemical bond;
Y is hydrogen, lower alkyl, aryl, aryl lower alkyl, lower alkenyl, lower alkynyl, halo, heterocyclic, heterocyclic lower alkyl, or lower alkyl heterocyclic, and is unsubstituted or substituted with at least one electron-withdrawing group and/or at least one electron-donating group, provided that when Y is halo, Z is a chemical bond, or
Z—Y taken together is NR₄NR₅R₇, NR₄OR₅, ONR₄R₇, OPR₄R₅, PR₄OR₅, SNR₄R₇, NR₄SR₇, SPR₄R₅, PR₄SR₇, NR₄PR₅R₆, PR₄NR₅R₇, N⁺R₅R₆R₇,

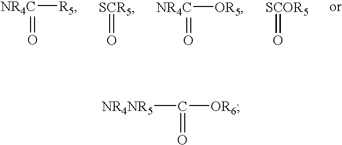

R′₆ is hydrogen, lower alkyl, lower alkenyl, or lower alkynyl, and is unsubstituted or substituted with at least one electron-withdrawing group or/and at least one electron-donating group;
R₄, R₅ and R₆ are independently hydrogen, lower alkyl, aryl, aryl lower alkyl, lower alkenyl, or lower alkynyl, and are each independently unsubstituted or substituted with at least one electron-withdrawing group or/and at least one electron-donating group;
R₇ is R₆, COOR₈, or COR₈, and is unsubstituted or substituted with at least one electron-withdrawing group or/and at least one electron-donating group;
R₈ is hydrogen, lower alkyl, or aryl lower alkyl, and is unsubstituted or substituted with at least one electron-withdrawing group or/and at least one electron-donating group;
n is 1-4; and
a is 1-3;
   or a pharmaceutically acceptable salt thereof; and a second agent effective in combination therewith to provide enhanced treatment of pain, by comparison with the first agent alone.

An illustrative compound of Formula (I) is lacosamide, (R)-2-acetamido-N-benzyl-3-methoxypropionamide.

In particular embodiments, the second agent comprises one or more drugs other than a compound of Formula (I), selected from analgesics, anticonvulsants, antidepressants and NMDA receptor antagonists.

Other embodiments, including particular aspects of the embodiments summarized above, will be evident from the detailed description that follows.

### DETAILED DESCRIPTION

Hovinga (2003) IDrugs 6(5):479-485, incorporated herein by reference in its entirety but not admitted to be prior art to the present invention, describes initial results of a trial of lacosamide in treatment of diabetic neuropathy.

McCleane et al. (2003) Neuroscience Letters 352:117-120, incorporated herein by reference in its entirety but not admitted to be prior art to the present invention, describe an open label 25-patient trial with lacosamide in subjects with resistant neuropathic pain, mainly radicular pain. None of the patients in this trial had diabetic neuropathy.

Therapeutic compositions and methods of use thereof are provided herein for treating diabetic pain such as pain associated with all types of painful diabetic neuropathy. In one embodiment, a therapeutic method is provided for treating pain in painful diabetic neuropathy, comprising administering a compound of Formula (I) or a pharmaceutically acceptable salt thereof, in combination with a second agent as described herein. In another embodiment, a therapeutic combination, for example in a form of a pharmaceutical composition, comprising a compound of Formula (I) or a pharmaceutically acceptable salt thereof and a second agent as described herein is useful for treating pain in painful diabetic neuropathy.

In a particular aspect, the painful diabetic neuropathy is associated with diabetes mellitus Type I or Type II, more particularly Type II. In a further aspect, the painful diabetic neuropathy is painful diabetic sensory polyneuropathy.

Pain in painful diabetic neuropathy has a number of different aspects. The compounds of Formula (I) therefore may also be used to treat an aspect of pain associated with painful diabetic neuropathy. Such aspects include, without limitation, average daily pain, overall pain, present pain intensity, pain interference with sleep, the subject's perception of pain interference with general activity, the subject's global impression of change in pain, clinical global impression of change in pain, the subject's perception of different neuropathic pain qualities, quality of life and proportion of pain-free days.

Unless the context demands otherwise, the term “treat,” “treating” or “treatment” herein includes preventive or prophylactic use of a combination, for example a combination of a first agent and second agent as defined herein, in a subject at risk of, or having a prognosis including, painful diabetic neuropathy, as well as use of such a combination in a subject already experiencing painful diabetic neuropathy, as a therapy to alleviate, relieve, reduce intensity of or eliminate the painful diabetic neuropathy or pain associated therewith or an underlying cause thereof.

The term “subject” refers to a warm-blooded animal, generally a mammal such as, for example, a cat, dog, horse, cow, pig, mouse, rat or primate, including a human. In one embodiment the subject is a human, for example a patient having painful diabetic neuropathy.

The first agent administered according to the present method comprises a compound of Formula (I) as set forth above, or a pharmaceutically acceptable salt thereof. Terms used in the description of Formula (I) and elsewhere in the present specification unless otherwise indicated, are defined as follows.

The term “alkyl,” alone or in combination with another term(s), means a straight- or branched-chain saturated hydrocarbyl substituent typically containing from 1 to about 20 carbon atoms, more typically from 1 to about 8 carbon atoms, and even more typically from 1 to about 6 carbon atoms.

The term “lower alkyl” refers to an alkyl substituent containing from 1 to 6 carbon atoms, especially 1 to 3 carbon atoms, that may be straight-chain or branched. Examples include methyl, ethyl, propyl, isopropyl, butyl, isobutyl, tertiary butyl, pentyl, hexyl, and the like, and isomers thereof.

The term “alkenyl,” alone or in combination with another term(s), means a straight- or branched-chain hydrocarbyl substituent containing one or more double bonds and typically from 2 to about 20 carbon atoms, more typically from 2 to about 8 carbon atoms, and even more typically from 2 to about 6 carbon atoms. Alkenyl groups, where asymmetric, can have cis or trans configuration.

The term “lower alkenyl” refers to an alkenyl substituent containing from 2 to 6 carbon atoms that may be straight-chained or branched and in the Z or E form. Examples include vinyl, propenyl, 1-butenyl, isobutenyl, 2-butenyl, 1-pentenyl, (Z)-2-pentenyl, (E)-2-pentenyl, (Z)-4-methyl-2-pentenyl, (E)-4-methyl-2-pentenyl, pentadienyl, e.g., 1, 3 or 2,4-pentadienyl, and the like.

The term “alkynyl,” alone or in combination with another term(s), means a straight- or branched-chain hydrocarbyl substituent containing one or more triple bonds and typically from 2 to about 20 carbon atoms, more typically from 2 to about 8 carbon atoms, and even more typically from 2 to about 6 carbon atoms.

The term “lower alkynyl” refers to an alkynyl substituent containing 2 to 6 carbon atoms that may be straight-chained or branched. It includes such groups as ethynyl, propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like.

The term “cycloalkyl,” alone or in combination with another term(s), means a completely or partially saturated alicyclic hydrocarbyl group containing from 3 to about 18 ring carbon atoms. Cycloalkyl groups may be monocyclic or polycyclic. Examples include cyclopropyl, cyclobutyl, cyclopentyl, cyclohexyl, cycloheptyl, cyclooctyl, cyclodecyl, cyclohexenyl, cyclopentenyl, cyclooctenyl, cycloheptenyl, decalinyl, hydroindanyl, indanyl, fenchyl, pinenyl, adamantyl, and the like. Cycloalkyl includes the cis or trans forms. Cycloalkyl groups may be unsubstituted or mono- or polysubstituted with electron withdrawing or/and electron donating groups as described below. Furthermore, the substituents may either be in endo- or exo-positions in bridged bicyclic systems. “Lower cycloalkyl” groups have 3 to 6 carbon atoms.

The term “alkoxy,” alone or in combination with another term(s), means an alkylether, i.e., —O-alkyl, substituent.

The term “lower alkoxy” refers to an alkoxy substituent containing from 1 to 6 carbon atoms, especially 1 to 3 carbon atoms, that may be straight-chain or branched. Examples include methoxy, ethoxy, propoxy, butoxy, isobutoxy, tert-butoxy, pentoxy, hexoxy and the like.

The term “aryl,” alone or in combination with another term(s), means an aromatic group which contains from about 6 to about 18 ring carbon atoms, and includes polynuclear aromatics. Aryl groups may be monocyclic or polycyclic, and optionally fused. A polynuclear aromatic group as used herein encompasses bicyclic and tricyclic fused aromatic ring systems containing from about 10 to about 18 ring carbon atoms. Aryl groups include phenyl, polynuclear aromatic groups (e.g., naphthyl, anthracenyl, phenanthrenyl, azulenyl and the like), and groups such as ferrocenyl. Aryl groups may be unsubstituted or mono- or polysubstituted with electron-withdrawing and/or electron-donating groups as described below.

“Aryl lower alkyl” groups include, for example, benzyl, phenylethyl, phenylpropyl, phenylisopropyl, phenylbutyl, diphenylmethyl, 1,1-diphenylethyl, 1,2-diphenylethyl, and the like.

The term “monosubstituted amino,” alone or in combination with another term(s), means an amino substituent wherein one of the hydrogen radicals is replaced by a non-hydrogen substituent. The term “disubstituted amino,” alone or in combination with another term(s), means an amino substituent wherein both of the hydrogen atoms are replaced by non-hydrogen substituents, which may be identical or different.

The term “halo” or “halogen” includes fluoro, chloro, bromo, and iodo.

The term “carbalkoxy” refers to —CO—O-alkyl, wherein alkyl may be lower alkyl as defined above.

The prefix “halo” indicates that the substituent to which the prefix is attached is substituted with one or more independently selected halogen radicals. For example, haloalkyl means an alkyl substituent wherein at least one hydrogen radical is replaced with a halogen radical. Examples of haloalkyl substituents include chloromethyl, 1-bromoethyl, fluoromethyl, difluoromethyl, trifluoromethyl, 1,1,1-trifluoroethyl, and the like. Illustrating further, “haloalkoxy” means an alkoxy substituent wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyloxy”), 1,1,1,-trifluoroethoxy, and the like. It should be recognized that if a substituent is substituted with more than one halogen radical, those halogen radicals may be identical or different, unless otherwise stated.

The term “acyl” includes alkanoyl containing from 1 to about 20 carbon atoms, preferably 1 to 6 carbon atoms, and may be straight-chain or branched. Acyl groups include, for example, formyl, acetyl, propionyl, butyryl, isobutyryl, tertiary butyryl, pentanoyl and isomers thereof, and hexanoyl and isomers thereof.

The terms “electron-withdrawing” and “electron-donating” refer to the ability of a substituent to withdraw or donate electrons, respectively, relative to that of hydrogen if a hydrogen atom occupied the same position in the molecule. These terms are well understood by one skilled in the art and are discussed, for example, in March (1985), Advanced Organic Chemistry, New York: John Wiley & Sons, at pp. 16-18, the disclosure of which is incorporated herein by reference. Electron-withdrawing groups include halo (including fluoro, chloro, bromo, and iodo), nitro, carboxy, lower alkenyl, lower alkynyl, formyl, carboxyamido, aryl, quaternary ammonium, haloalkyl (such as trifluoromethyl), aryl lower alkanoyl, carbalkoxy, and the like. Electron-donating groups include hydroxy, lower alkoxy (including methoxy, ethoxy, and the like), lower alkyl (including methyl, ethyl, and the like), amino, lower alkylamino, di(lower alkyl)amino, aryloxy (such as phenoxy), mercapto, lower alkylthio, lower alkylmercapto, disulfide (lower alkyldithio), and the like. One of ordinary skill in the art will appreciate that some of the aforesaid substituents may be considered to be electron-donating or electron-withdrawing under different chemical conditions. Moreover, the present invention contemplates any combination of substituents selected from the above-identified groups.

The term “heterocyclic” means a ring substituent that contains one or more sulfur, nitrogen and/or oxygen ring atoms. Heterocyclic groups include heteroaromatic groups and saturated and partially saturated heterocyclic groups. Heterocyclic groups may be monocyclic, bicyclic, tricyclic or polycyclic and can be fused rings. They typically contain up to 18 ring atoms, including up to 17 ring carbon atoms, and can contain in total up to about 25 carbon atoms, but preferably are 5- to 6-membered rings. Heterocyclic groups also include the so-called benzoheterocyclics. Representative heterocyclic groups include furyl, thienyl, pyrazolyl, pyrrolyl, methylpyrrolyl, imidazolyl, indolyl, thiazolyl, oxazolyl, isothiazolyl, isoxazolyl, piperidyl, pyrrolinyl, piperazinyl, quinolyl, triazolyl, tetrazolyl, isoquinolyl, benzofuryl, benzothienyl, morpholinyl, benzoxazolyl, tetrahydrofuryl, pyranyl, indazolyl, purinyl, indolinyl, pyrazolindinyl, imidazolinyl, imadazolindinyl, pyrrolidinyl, furazanyl, N-methylindolyl, methylfuryl, pyridazinyl, pyrimidinyl, pyrazinyl, pyridyl, epoxy, aziridino, oxetanyl, and azetidinyl groups, as well as N-oxides of nitrogen-containing heterocyclics, such as the N-oxides of pyridyl, pyrazinyl, and pyrimidinyl groups and the like. Heterocyclic groups may be unsubstituted or mono- or polysubstituted with electron-withdrawing and/or electron-donating groups.

In one embodiment, a heterocyclic group is selected from thienyl, furyl, pyrrolyl, benzofuryl, benzothienyl, indolyl, methylpyrrolyl, morpholinyl, pyridyl, pyrazinyl, imidazolyl, pyrimidinyl, and pyridazinyl, especially furyl, pyridyl, pyrazinyl, imidazolyl, pyrimidinyl, and pyridazinyl, more especially furyl and pyridyl.

In another embodiment, a heterocyclic group is selected from furyl, optionally substituted with at least one lower alkyl group (preferably one having 1-3 carbon atoms, for example methyl), pyrrolyl, imidazolyl, pyridyl, pyrazinyl, pyrimidinyl, oxazolyl and thiazolyl, especially furyl, pyridyl, pyrazinyl, pyrimidinyl, oxazolyl and thiazolyl, more especially furyl, pyridyl, pyrimidinyl and oxazolyl.

Illustratively, in the compound of Formula (I) n is 1, but di- (n=2), tri- (n=3) and tetrapeptides (n=4) are also contemplated to be useful herein.

R in the compound of Formula (I) is illustratively aryl lower alkyl, especially benzyl where the phenyl ring thereof is unsubstituted or substituted with one or more electron-donating groups and/or electron-withdrawing groups, such as halo (e.g., fluoro).

R₁ in the compound of Formula (I) is preferably hydrogen or lower alkyl, especially methyl.

Particularly suitable electron-withdrawing and/or electron-donating substituents are halo, nitro, alkanoyl, formyl, arylalkanoyl, aryloyl, carboxyl, carbalkoxy, carboxamido, cyano, sulfonyl, sulfoxide, heterocyclic, guanidine, quaternary ammonium, lower alkenyl, lower alkynyl, sulfonium salts, hydroxy, lower alkoxy, lower alkyl, amino, lower alkylamino, di(lower alkyl)amino, amino lower alkyl, mercapto, mercaptoalkyl, alkylthio, and alkyldithio. The term “sulfide” encompasses mercapto, mercapto alkyl and alkylthio, while the term disulfide encompasses alkylthio. Preferred electron-withdrawing and/or electron-donating groups are halo and lower alkoxy, especially fluoro and methoxy. These preferred substituents may be present in any one or more of the groups R, R₁, R₂, R₃, R₄, R₅, R₆, R′₆, R₇ or R₈ as defined herein.

Z—Y groups representative of R₂ and/or R₃ include hydroxy, alkoxy (such as methoxy and ethoxy), aryloxy (such as phenoxy), thioalkoxy (such as thiomethoxy and thioethoxy), thioaryloxy (such as thiophenoxy), amino, alkylamino (such as methylamino and ethylamino), arylamino (such as anilino), lower dialkylamino (such as dimethylamino), trialkylammonium salt, hydrazino, alkylhydrazino and arylhydrazino (such as N-methylhydrazino and N-phenylhydrazino), carbalkoxy hydrazino, aralkoxycarbonyl hydrazino, aryloxycarbonyl hydrazino, hydroxylamino (such as N-hydroxylamino (—NHOH)), lower alkoxyamino (NHOR₁₈ wherein R₁₈ is lower alkyl, e.g., methyl), N-lower alkylhydroxylamino (N(R₁₈)OH wherein R₁₈ is lower alkyl), N-lower alkyl-O-lower alkylhydroxylamino (N(R₁₈)OR₁₉ wherein R₁₈ and R₁₉ are independently lower alkyl), and o-hydroxylamino (—O—NH₂)), alkylamido (such as acetamido), trifluoroacetamido, and heterocyclylamino (such as pyrazoylamino).

Preferred heterocyclic groups representative of R₂ and/or R₃ are monocyclic 5- or 6-membered heterocyclic moieties of the formula

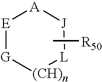

including unsaturated, partially and fully saturated forms thereof, wherein n is 0 or 1; R₅₀ is hydrogen or an electron-withdrawing or electron-donating group; A, E, L, J and G are independently CH, or a heteroatom selected from the group consisting of N, O and S; but when n is 0, G is CH, or a heteroatom selected from the group consisting of N, O and S; with the proviso that at most two of A, E, L, J and G are heteroatoms.

If n is 0, the above monocyclic heterocyclic ring is 5-membered, while if n is 1, the ring is 6-membered.

If the ring depicted hereinabove contains a nitrogen ring atom, then the N-oxide forms are also contemplated to be within the scope of the invention.

When R₂ or R₃ comprises a heterocyclic group of the above formula, it may be bonded to the main chain by a ring carbon atom. When n is 0, R₂ or R₃ may additionally be bonded to the main chain by a nitrogen ring atom.

Other preferred moieties of R₂ and R₃ are hydrogen, aryl (e.g., phenyl), arylalkyl (e.g., benzyl), and alkyl. Such moieties can be unsubstituted or mono- or polysubstituted with electron-withdrawing and/or electron-donating groups. In various embodiments, R₂ and R₃ are independently hydrogen; lower alkyl, either unsubstituted or substituted with one or more electron-withdrawing and/or electron-donating groups such as lower alkoxy (e.g., methoxy, ethoxy, and the like); N-hydroxylamino; N-lower alkylhydroxyamino; N-lower alkyl-O-lower alkyl; or alkylhydroxylamino.

In some embodiments, one of R₂ and R₃ is hydrogen.

In one embodiment n in Formula (I) is 1 and one of R₂ and R₃ is hydrogen. Illustratively in this embodiment, R₂ is hydrogen and R₃ is lower alkyl or Z—Y where Z is O, NR₄ or PR₄, and Y is hydrogen or lower alkyl; or Z—Y is NR₄NR₅R₇, NR₄OR₅, ONR₄R₇,

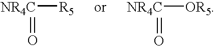

In another embodiment, n is 1, R₂ is hydrogen, and R₃ is lower alkyl which is unsubstituted or substituted with an electron-withdrawing or electron-donating group, NR₄OR₅, or ONR₄R₇.

In yet another embodiment,
n is 1;
R is aryl lower alkyl, which aryl group is unsubstituted or substituted with an electron-withdrawing group, for example aryl can be phenyl, which is unsubstituted or substituted with halo;
R₁ is lower alkyl;
R₂ is hydrogen; and
R₃ is lower alkyl which is unsubstituted or substituted with hydroxy, lower alkoxy, NR₄OR₅ or ONR₄R₇, wherein R₄, R₅ and R₇ are independently hydrogen or lower alkyl.

In yet another embodiment, R₂ is hydrogen and R₃ is hydrogen, an alkyl group which is unsubstituted or substituted with at least one electron-withdrawing or electron-donating group or Z—Y. In this embodiment, R₃ is illustratively hydrogen, an alkyl group such as methyl, which is unsubstituted or substituted with an electron-donating group such as lower alkoxy, more especially methoxy or ethoxy, or with NR₄OR₅ or ONR₄R₇, wherein R₄, R₅ and R₇ are independently hydrogen or lower alkyl.

In yet another embodiment, R₂ and R₃ are independently hydrogen, lower alkyl, or Z—Y; Z is O, NR₄ or PR₄; Y is hydrogen or lower alkyl; or Z—Y is NR₄NR₅R₇, NR₄OR₅, ONR₄R₇,

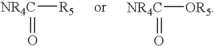

It is preferred that R is aryl lower alkyl. The most preferred aryl for R is phenyl. The most preferred R group is benzyl. The aryl group is unsubstituted or substituted with an electron-withdrawing or electron-donating group. If the aryl ring in R is substituted, it is most preferred that it is substituted with an electron-withdrawing group, The most preferred electron-withdrawing group for R is halo, especially fluoro.

The preferred R₁ is lower alkyl, especially methyl.

In one embodiment R is aryl lower alkyl, e.g., benzyl, and R₁ is lower alkyl, e.g., methyl.

Further preferred compounds are compounds of Formula (I) wherein
n is 1;
R is aryl or aryl lower alkyl, such as benzyl, wherein the aryl group is unsubstituted or substituted with an electron-withdrawing or electron-donating group;
R₁ is lower alkyl;
R₂ is hydrogen; and
R₃ is hydrogen, a lower alkyl group, especially methyl which is substituted with an electron-withdrawing or electron-donating group, or Z—Y.
   In this embodiment, it is more preferred that R₃ is hydrogen, a lower alkyl group, especially methyl, which may be substituted with an electron-donating group such as lower alkoxy (e.g., methoxy, ethoxy or the like), NR₄OR₅ or ONR₄R₇ wherein these groups are as defined hereinabove.

In one aspect, the compound is represented by Formula (II)

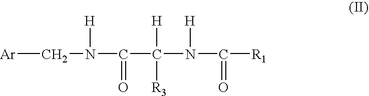

or a pharmaceutically acceptable salt thereof, wherein
Ar is aryl, especially phenyl, which is unsubstituted or substituted with at least one halo;
R₁ is lower alkyl, especially C₁₋₃ alkyl, for example methyl; and
R₃ is hydrogen or lower alkyl, which is unsubstituted or substituted with at least one electron-withdrawing or electron-donating group or Z—Y; for example R₃ is —CH₂-Q, wherein Q is lower alkoxy, especially C₁₋₃ alkoxy, for example methoxy.

In another aspect, the compound has formula (I) wherein
n is 1;
R is unsubstituted or substituted benzyl, in particular halo-substituted benzyl;
R₁ is lower alkyl, especially C₁₋₃ alkyl, for example methyl;
R₂ is hydrogen; and
R₃ is as broadly defined herein.

In yet another aspect, the compound is represented by Formula (III)

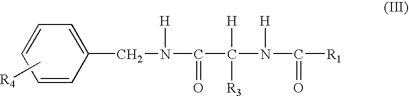

or a pharmaceutically acceptable salt thereof, wherein
R₄ is one or more substituents independently selected from the group consisting of hydrogen, halo, alkyl, alkenyl, alkynyl, nitro, carboxy, formyl, carboxyamido, aryl, quaternary ammonium, haloalkyl, aryl alkanoyl, hydroxy, alkoxy, amino, alkylamino, dialkylamino, aryloxy, mercapto, alkylthio, alkylmercapto, and disulfide;
R₃ is selected from the group consisting of hydrogen, alkyl, alkoxy, alkoxyalkyl, aryl, N-alkoxy-N-alkylamino, and N-alkoxyamino; and
R₁ is alkyl.

Alkyl, alkoxy, alkenyl and alkynyl groups in a compound of Formula (III) are lower alkyl, alkoxy, alkenyl and alkynyl groups having no more than 6, more typically no more than 3, carbon atoms.

In a particular aspect, R₄ substituents in a compound of Formula (III) are independently selected from hydrogen and halo, more particularly fluoro, substituents.

In a particular aspect, R₃ in a compound of Formula (III) is alkoxyalkyl, phenyl, N-alkoxy-N-alkylamino or N-alkoxyamino.

In a particular aspect, R₁ in a compound of Formula (III) is C₁₋₃ alkyl.

In a more particular aspect, no more than one R₄ substituent is fluoro and all others are hydrogen; R₃ is selected from the group consisting of methoxymethyl, phenyl, N-methoxy-N-methylamino and N-methoxyamino; and R₁ is methyl.

It is to be understood that combinations and permutations of R₁, R₂, R₃ and R groups and values of n, even if such combinations and permutations are not explicitly described herein, are contemplated to be within the scope of the present invention. Moreover, the present invention also encompasses therapeutic combinations that comprise a compound having one or more elements of each of the Markush groupings described for R₁, R₂, R₃ and R and the various combinations thereof. Thus, for example, the present invention contemplates that R₁ and R may independently be one or more of the substituents listed hereinabove in combination with any of the R₂ and R₃ substituents, independently with respect to each of the n

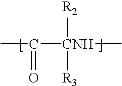

subunits of the compound of Formula (I).

Compounds useful herein may contain one or more asymmetric carbons and may exist in optically active forms. The configuration around each asymmetric carbon can be either the D or L configuration. Configuration around a chiral carbon atom can also be described as R or S in the Cahn-Prelog-Ingold system. All of the various configurations around each asymmetric carbon, including the various enantiomers and diastereomers as well as mixtures of enantiomers, diastereomers or both, including but not limited to racemic mixtures, are contemplated by the present invention.

More particularly, in a compound of Formula (I) where R₂ and R₃ are not identical, there exists asymmetry at the carbon atom to which the groups R₂ and R₃ are attached. As used herein, the term “configuration” generally refers to the configuration around the carbon atom to which R₂ and R₃ are attached, even though other chiral centers may be present in the molecule. Therefore, unless the context demands otherwise, when referring to a particular configuration such as D or L, it is to be understood to mean the D- or L-stereoisomer at the carbon atom to which R₂ and R₃ are attached. However, all possible enantiomers and diastereomers at other chiral centers, if any, present in the compound are encompassed herein.

The compounds useful herein can comprise the L- or D-stereoisomer as defined above, or any mixture thereof, including without limitation a racemic mixture. The D-stereoisomer is generally preferred. In lacosamide, the D-stereoisomer corresponds to the R-enantiomer according to R,S terminology.

In one embodiment the compound, for example lacosamide, is substantially enantiopure. As used herein, the term “substantially enantiopure” means having at least 88%, preferably at least 90%, more preferably at least 95%, 96%, 97%, 98% or 99% enantiomeric purity.

Illustrative compounds that can be used in the present combination include:
(R)-2-acetamido-N-benzyl-3-methoxypropionamide (lacosamide);
(R)-2-acetamido-N-benzyl-3-ethoxypropionamide;
O-methyl-N-acetyl-D-serine-m-fluorobenzylamide;
O-methyl-N-acetyl-D-serine-p-fluorobenzylamide;
N-acetyl-D-phenylglycinebenzylamide;
D-1,2-(N,O-dimethylhydroxylamino)-2-acetamido acetic acid benzylamide; and
D-1,2-(O-methylhydroxylamino)-2-acetamido acetic acid benzylamide.

Depending upon the substituents, certain of the present compounds may form salts. For example, compounds of Formulas (I), (II) and (III) can form salts with a wide variety of acids, inorganic and organic, including pharmaceutically acceptable acids. Such salts can have enhanced water solubility and may be particularly useful in preparing pharmaceutical compositions for use in situations where enhanced water solubility is advantageous.

Pharmaceutically acceptable salts are those having therapeutic efficacy without unacceptable toxicity. Salts of inorganic acids such as hydrochloric, hydroiodic, hydrobromic, phosphoric, metaphosphoric, nitric and sulfuric acids as well as salts of organic acids such as tartaric, acetic, citric, malic, benzoic, perchloric, glycolic, gluconic, succinic, arylsulfonic (e.g., p-toluene sulfonic, benzenesulfonic), phosphoric and malonic acids and the like, can be used.

Compounds useful herein can be prepared by any known procedure of synthesis, for example as described in above-referenced U.S. Pat. No. 5,378,729 and No. 5,773,475, each of which is incorporated herein by reference.

A compound as described herein is used in a therapeutically effective amount. A physician can determine a suitable dosage of a compound, which can vary with the particular compound chosen, the route and method of administration, and the age and other characteristics of the individual patient. The physician can initiate treatment with small doses, for example substantially less than an optimum dose of the compound, and increase the dose by small increments until an optimum effect under the circumstances is achieved. When the composition is administered orally, larger quantities of the compound may be required to produce the same therapeutic benefit as a smaller quantity given parenterally.

In a particular aspect, the compound, for example lacosamide, is administered in an amount ranging from about 1 mg to about 10 mg per kilogram of body weight per day. Typically a patient can be treated with the compound, for example lacosamide, at a dose of at least about 50 mg/day, for example at least about 100 mg/day, at least about 200 mg/day, at least about 300 mg/day or at least about 400 mg/day. Generally, a suitable dose is not greater than about 6 g/day, for example not greater than about 1 g/day or not greater than about 600 mg/day. In some cases, however, higher or lower doses may be needed.

In another aspect, the daily dose is increased until a predetermined daily dose is reached which is maintained during further treatment.

In yet another aspect, several divided doses are administered daily. For example, no more than three doses per day, or no more than two doses per day, may be administered. However, it is often most convenient to administer no more than a single dose per day.

Doses expressed herein on a daily basis, for example in mg/day, are not to be interpreted as requiring a once-a-day frequency of administration. For example, a dose of 300 mg/day can be given as 100 mg three times a day, or as 600 mg every second day.

In yet another aspect, an amount of the compound, for example lacosamide, is administered which results in a plasma concentration of the compound of about 0.1 to about 15 μg/ml (trough) and about 5 to about 18.5 μg/ml (peak), calculated as an average over a plurality of treated subjects.

The compound of Formulas (I), (II) or (III), for example lacosamide, can be administered in any convenient and effective manner, such as by oral, intravenous, intraperitoneal, intramuscular, intrathecal, subcutaneous or transmucosal (e.g., buccal) routes. Oral or intravenous administration is generally preferred.

For oral administration, the compound is typically administered as a component of an orally deliverable pharmaceutical composition that further comprises an inert diluent or an assimilable edible carrier, or it may be incorporated into the subject's food. In an orally deliverable pharmaceutical composition, the compound can be incorporated together with one or more excipients and administered in the form of tablets, troches, pills, capsules, elixirs, suspensions, syrups, wafers, or the like. Such compositions typically contain at least about 1%, more typically about 5% to about 80%, by weight of the compound, for example lacosamide. The amount of the compound in the composition is such that, upon administration of the composition, a suitable dosage as set forth above can conveniently be provided. Illustratively, a pharmaceutical composition useful for oral delivery of a compound of Formulas (I), (II) or (III), for example lacosamide, contains about 10 mg to about 6 g, for example about 50 to about 1000 mg, or about 100 to about 600 mg, of the compound.

In particular embodiments the composition is enclosed in hard or soft shell (e.g., gelatin) capsules, or is in a form of compressed or molded tablets. The composition illustratively comprises as excipients one or more of a diluent such as lactose or dicalcium phosphate (in the case of capsules a liquid carrier can be present); a binding agent such as gum tragacanth, acacia, corn starch or gelatin; a disintegrating agent such as corn starch, potato starch, alginic acid or the like; a lubricant such as magnesium stearate; and a sweetening agent such as sucrose or saccharin and/or a flavoring agent such as peppermint, oil of wintergreen, or cherry flavoring can be added if desired.

Various other excipients may be present as coatings or otherwise modifying the physical form of the composition. For example, tablets, pills, or capsules may be coated with shellac, sugar or both. A syrup or elixir may contain the active compound, sucrose as a sweetening agent, methyl- and propylparabens as preservatives, a dye, and flavoring such as cherry or orange flavor. The active compound can be incorporated into a sustained-release formulation. For example, sustained-release dosage forms are contemplated wherein the compound is bound to an ion exchange resin which, optionally, can be coated with a diffusion barrier coating to modify the release properties of the resin.

Pharmaceutical compositions suitable for injectable use include sterile aqueous solutions (where the compound is water soluble), dispersions, and sterile powders for extemporaneous preparation of sterile injectable solutions or dispersions. In such cases the injectable composition must be sterile and must be sufficiently fluid to permit easy syringeability. The composition must be stable under the conditions of manufacture and storage and must typically be preserved against the contaminating action of microorganisms such as bacteria and fungi. The carrier can be a solvent or dispersion medium containing, for example, water, ethanol, polyol (for example, glycerol, propylene glycol, liquid polyethylene glycol, or the like), suitable mixtures thereof, and vegetable oils. Proper fluidity can be maintained, for example, by use of a coating such as lecithin, by maintenance of a required particle size in the case of dispersions, and by use of surfactants. Microbial action can be inhibited by various antibacterial and antifungal agents, for example, parabens, chlorobutanol, phenol, sorbic acid, thimerosal, or the like. In many cases, it will be preferable to include tonicity agents, for example, sugars or sodium chloride, to provide a substantially isotonic liquid for injection. Prolonged absorption of injectable compositions can be brought about by use in the compositions of agents delaying absorption, for example aluminum monostearate or gelatin.

Sterile injectable solutions can be prepared by incorporating the active compound in a required amount in an appropriate solvent with various of the other ingredients mentioned above, as required, followed by filtered sterilization. Generally, dispersions are prepared by incorporating sterilized active compound into a sterile vehicle which contains the dispersion medium and other excipient ingredients such as those mentioned above. Sterile powders for preparation of sterile injectable solutions can be prepared by vacuum-drying or freeze-drying a previously sterile-filtered solution or dispersion.

It is especially advantageous to formulate parenteral compositions in dosage unit form for ease of administration and uniformity of dosage. “Dosage unit form” as used herein refers to physically discrete units suited as unitary dosages for the subject to be treated, each unit containing a predetermined quantity of active agent(s) calculated to produce a desired therapeutic effect in association with the pharmaceutical carrier. The specifics for dosage unit forms of the invention are dictated by and directly dependent on (a) the unique characteristics of the active agent(s) and the particular therapeutic effect to be achieved, and (b) the limitations in the art of compounding such active agents for treatment of disease in a living subject having a condition in which bodily health is impaired as herein disclosed in detail.

As used herein, “pharmaceutically acceptable carrier” includes any and all solvents, dispersion media, coatings, antibacterial and antifungal agent, isotonic and absorption delaying agents for pharmaceutically active substances as known in the art. Except insofar as any carrier substance is incompatible with an active ingredient, its use in the present therapeutic compositions is contemplated.

The active agent(s) can be compounded for convenient administration in effective amounts with a suitable pharmaceutically acceptable carrier in dosage unit form as hereinbefore described. A unit dosage form can, for example, contain a compound of Formulas (I), (II) or (III), for example lacosamide, in amounts ranging from about 10 mg to about 6 g. Expressed in proportions, a compound of Formulas (I), (II) or (III), for example lacosamide, is generally present in about 1 to about 750 mg/ml of carrier. Dosages of one or more drugs present as the second agent herein are determined by reference to the usual dose and manner of administration of such drugs.

The present method comprises administering, in combination with a first agent as described above, a second agent effective in combination with the first agent to provide enhanced treatment of pain, by comparison with the first agent alone.

“Enhanced treatment of pain” in the present context means that the combination is superior to the first agent alone in at least one of the following respects:
(a) greater reduction of intensity and/or duration of pain;
(b) enabling dose reduction of either the first agent or the second agent or both by comparison with a typical effective dose when used in monotherapy;
(c) reduction in adverse side effects; and/or
(d) improved therapeutic ratio.
   It is not required that the first agent and the second agent interact more than additively, but in some cases the reduction of intensity and/or duration of pain provided by the combination can be greater than would be expected based on the effectiveness of either agent alone at the same dose.

The term “therapeutic combination” refers to a plurality of agents that, when administered to a subject together or separately, are co-active in bringing therapeutic benefit to the subject. Such administration is referred to as “combination therapy,” “co-therapy,” “adjunctive therapy” or “add-on therapy.” For example, one agent can potentiate or enhance the therapeutic effect of another, or reduce an adverse side effect of another, or one or more agents can be effectively administered at a lower dose than when used alone, or can provide greater therapeutic benefit than when used alone, or can complementarily address different aspects, symptoms or etiological factors of a disease or condition.

In various non-limiting embodiments, the second agent comprises at least one drug other than a compound of Formula (I) selected from the group consisting of analgesics, anticonvulsants, antidepressants and NMDA receptor antagonists.

In one aspect, the method comprises administering, in combination or adjunctive therapy with a first agent comprising a compound of Formulas (I), (II) or (III), for example lacosamide, at least one analgesic. Suitable analgesics include opioid and non-opioid analgesics.

Nonlimiting examples of opioid and non-opioid analgesics that can be useful in combination or adjunctive therapy with a compound of Formulas (I), (II) or (III), e.g., lacosamide, include without limitation acetaminophen, alfentanil, allylprodine, alphaprodine, anileridine, benzylmorphine, bezitramide, buprenorphine, butorphanol, clonitazene, codeine, cyclazocine, desomorphine, dextromoramide, dextropropoxyphene, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacyl-morphan, lofentanil, meperidine, meptazinol, metazocine, methadone, metopon, morphine, myrophine, nalbuphine, nalorphine, narceine, nicomorphine, norlevorphanol, normethadone, normorphine, norpipanone, opium, oxycodone, oxymorphone, papaveretum, pentazocine, phenadoxone, phenazocine, phenomorphan, phenoperidine, piminodine, piritramide, proheptazine, promedol, properidine, propiram, propoxyphene, sufentanil, tilidine, tramadol, NO-naproxen, NCX-701, ALGRX-4975, pharmaceutically acceptable salts thereof, and combinations thereof. In an illustrative example, the second agent comprises an opioid analgesic such as morphine or a pharmaceutically acceptable salt thereof.

In another aspect, the method comprises administering, in combination or adjunctive therapy with a first agent comprising a compound of Formulas (I), (II) or (III), for example lacosamide, at least one anticonvulsant.

Nonlimiting examples of anticonvulsants that can be useful in combination or adjunctive therapy with a compound of Formulas (I), (II) or (III), e.g., lacosamide, include without limitation acetylpheneturide, albutoin, aminoglutethimide, 4-amino-3-hydroxybutyric acid, atrolactamide, beclamide, buramate, carbamazepine, cinromide, clomethiazole, clonazepam, decimemide, diethadione, dimethadione, doxenitoin, eterobarb, ethadione, ethosuximide, ethotoin, felbamate, fluoresone, fosphenytoin, gabapentin, ganaxolone, lamotrigine, levetiracetam, lorazepam, mephenyloin, mephobarbital, metharbital, methetoin, methsuximide, midazolam, narcobarbital, nitrazepam, oxcarbazepine, paramethadione, phenacemide, phenetharbital, pheneturide, phenobarbital, phensuximide, phenylmethylbarbituric acid, phenyloin, phenethylate, pregabalin, primidone, progabide, remacemide, rufinamide, suclofenide, sulthiame, talampanel, tetrantoin, tiagabine, topiramate, trimethadione, valproic acid, valpromide, vigabatrin, zonisamide, pharmaceutically acceptable salts thereof, and combinations thereof.

Illustratively, the second agent comprises one or more of carbamazepine, phenyloin, gabapentin, pregabalin, lamotrigine, levetiracetam, and pharmaceutically acceptable salts thereof. In an illustrative example, the second agent comprises gabapentin.

In yet another aspect, the method comprises administering, in combination or adjunctive therapy with a first agent comprising a compound of Formulas (I), (II) or (III), for example lacosamide, at least one antidepressant.

Nonlimiting examples of antidepressants that can be useful in combination or adjunctive therapy with a compound of Formulas (I), (II) or (III), e.g., lacosamide, include without limitation bicyclic, tricyclic and tetracyclic antidepressants, hydrazides, hydrazines, phenyloxazolidinones and pyrrolidones. Specific examples include adinazolam, adrafinil, amineptine, amitriptyline, amitriptylinoxide, amoxapine, befloxatone, bupropion, butacetin, butriptyline, caroxazone, citalopram, clomipramine, cotinine, demexiptiline, desipramine, dibenzepin, dimetacrine, dimethazan, dioxadrol, dothiepin, doxepin, duloxetine, etoperidone, femoxetine, fencamine, fenpentadiol, fluacizine, fluoxetine, fluvoxamine, hematoporphyrin, hypericin, imipramine, imipramine N-oxide, indalpine, indeloxazine, iprindole, iproclozide, iproniazid, isocarboxazid, levophacetoperane, lofepramine, maprotiline, medifoxamine, melitracen, metapramine, metralindole, mianserin, milnacipran, minaprine, mirtazapine, moclobemide, nefazodone, nefopam, nialamide, nomifensine, nortriptyline, noxiptilin, octamoxin, opipramol, oxaflozane, oxitriptan, oxypertine, paroxetine, phenelzine, piberaline, pizotyline, prolintane, propizepine, protriptyline, pyrisuccideanol, quinupramine, reboxetine, ritanserin, roxindole, rubidium chloride, sertraline, sulpiride, tandospirone, thiazesim, thozalinone, tianeptine, tofenacin, toloxatone, tranylcypromine, trazodone, trimipramine, tryptophan, venlafaxine, viloxazine, zimeldine, pharmaceutically acceptable salts thereof, and combinations thereof. In a particular aspect, the second agent comprises duloxetine.

In yet another aspect, the method comprises administering, in combination or adjunctive therapy with a first agent comprising a compound of Formulas (I), (II) or (III), for example lacosamide, at least one NMDA receptor antagonist.

Nonlimiting examples of NMDA receptor antagonists that can be useful in combination or adjunctive therapy with a compound of Formulas (I), (II) or (III), e.g., lacosamide, include without limitation amantadine, D-AP5, aptiganel, CPP, dexanabinol, dextromethorphan, dextropropoxyphene, 5,7-dichlorokynurenic acid, gavestinel, ifendopril, ketamine, ketobemidone, licostinel, LY-235959, memantine, methadone, MK-801, phencyclidine, remacemide, selfotel, tiletamine, pharmaceutically acceptable salts thereof, and combinations thereof. In a particular aspect, the second agent comprises memantine.

Suitable regimens including doses and routes of administration for particular second agents can be determined from readily-available reference sources relating to these agents, for example Physicians' Desk Reference (PDR), 60th edition, Montvale, N.J.: Thomson (2006) and various internet sources known to those of skill in the art. When administered in combination or adjunctive therapy with a compound of Formulas (I), (II) or (III), for example lacosamide, the second agent can be used at a full dose, but the physician may elect to administer less than a full dose of the second agent, at least initially.

The compound of Formulas (I), (II) or (III), for example lacosamide, and the second agent can be provided in one pharmaceutical preparation (single dosage form) for administration to the subject at the same time, or in two or more distinct preparations (separate dosage forms) for administration to the subject at the same or different times, e.g., sequentially, and/or at the same or different frequencies. The two distinct preparations can be provided in forms adapted for administration by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in a first container, a first agent comprising a compound of Formulas (I), (II) or (III), for example lacosamide, and, in a second container, a second agent as described herein. In another example, the first agent and second agent are separately packaged and available for sale independently of one another, but are co-marketed or co-promoted for use according to the invention. The separate dosage forms may also be presented to a subject separately and independently, for use according to the invention.

Depending on the dosage forms, which may be identical or different, e.g., fast release dosage forms, controlled release dosage forms or depot forms, the first agent and second agent may be administered on the same or on different schedules, for example on a daily, weekly or monthly basis.

In one aspect, a therapeutic combination as described herein is effective for treating pain associated with painful diabetic distal sensory polyneuropathy.

In a further aspect, the therapeutic combination is effective for treatment of an aspect of pain. Nonlimiting examples of such aspects include average daily pain, overall pain, present pain intensity, pain interference with sleep, the subject's perception of pain interference with general activity, the subject's global impression of change in pain, clinical global impression of change in pain, the subject's perception of different neuropathic pain qualities, quality of life and proportion of pain-free days.

In yet another aspect, the therapeutic combination is useful for treating pain in painful diabetic neuropathy which is associated with diabetes mellitus Type I or Type II. In a particular aspect, the painful diabetic neuropathy is associated with diabetes mellitus Type II.

In another embodiment, a method is provided for treating pain in painful diabetic neuropathy comprising administering a therapeutic combination as described herein (a “primary combination”), in further combination with an active agent effective for treating diabetes mellitus Type I or Type II, more particularly Type II. The active agent effective for treating diabetes mellitus may be administered together with one or more of the components of the primary combination, for example in a single dosage form, or separately, i.e., in a dosage form separate from the components of the primary combination. Thus, a pharmaceutical composition of the present invention may comprise a first agent and second agent as described herein, and further comprise an active agent effective for treating diabetes mellitus Type I or Type II, more particularly Type II.

The active agent for treating Diabetes mellitus Type I or Type II, preferably Type II, is preferably an agent which does not induce weight gain in the subject.

### EXAMPLES

### Example 1

The following example shows the properties of lacosamide in reducing pain in a clinical trial in subjects with painful diabetic neuropathy, in particular with diabetic distal sensory polyneuropathy.

A randomized, double-blind placebo controlled trial to investigate safety and efficacy of lacosamide in painful diabetic neuropathy was conducted.

### Objectives

The primary objective of the study was to determine whether lacosamide was effective in reducing pain in subjects with diabetic distal sensory polyneuropathy. Secondary objectives were the following:
to investigate how lacosamide affects different qualities of neuropathic pain;
to investigate whether lacosamide affects sleep and activity in subjects suffering from diabetic distal sensory polyneuropathy;
to investigate whether lacosamide influences Quality of Life and Profile of Mood States;
to further investigate tolerability and safety of lacosamide;
to investigate pre- and post-dose plasma concentrations of unchanged lacosamide.

### Methodology

This was a multicenter, double-blind, placebo-controlled trial to assess the efficacy, safety, tolerability, and pre- and post-dose plasma concentrations of oral lacosamide in subjects with painful diabetic neuropathy.

Baseline data were collected during the last week of a 4-week Run-In Phase to ensure subject eligibility. Eligible subjects were then randomized to receive a maximum of 400 mg/day of lacosamide (starting at 100 mg/day for 3 weeks, then titrating up at 100 mg intervals for 3 weeks) or placebo. The highest attained dose was maintained for 4 weeks during the Maintenance Phase, after which subjects entered the Taper Phase and were tapered off of study medication for 1 week. The Taper Phase was followed by a 2-week Safety Follow-Up Phase.

### Number of Subjects (Planned and Analyzed)

A total of 140 subjects were planned to be enrolled in order to achieve 100 evaluable subjects.

A total of 438 subjects were screened for this trial. Two hundred seventy-seven were screen failures due to stringent entry criteria, and 42 were Run-In failures. Therefore, a total of 119 subjects were randomized. All 119 subjects who were randomized also received at least one dose of trial medication and are referred to as the Safety Set (SS). All randomized subjects also had at least one post-baseline efficacy assessment and are considered part of the Full Analysis Set (FAS). Ninety-three subjects in the FAS completed the Maintenance Phase and did not have a major protocol violation and are, therefore, considered part of the Per Protocol Set (PPS). A total of 94 subjects completed all phases of the trial.

### Diagnosis and Main Criteria for Inclusion

Subjects were male or female, age 18 or older. Subjects had clinically diagnosed pain attributed to diabetic distal sensory polyneuropathy for 1-5 years and a diagnosis of diabetes mellitus (Type I or Type II). Subjects had at least moderate pain (mean pain intensity during the Baseline week ≧4 out of 10 on an 11-point Likert scale) which had been stable for 4 weeks prior to randomization. Furthermore, subjects had good or fair diabetic control (glycosylated hemoglobin (HbA_{1C}) <10%), which was optimized (best effort to achieve best control) for at least three months prior to Visit 1. Test product, dose and mode of administration, batch number

Subjects took 50 mg and 100 mg lacosamide tablets (Schwarz Pharma AG, Germany).

### Duration of Treatment

After completion of screening assessments, subjects began a 4-week Run-In Phase. Eligible subjects were randomized at Visit 3 in a 1:1 ratio to active lacosamide or matching placebo starting with 100 mg/day (50 mg twice daily (BID)) for three weeks. Following the titration scheme, their dosage was escalated by 100 mg in weekly increments to a maximum dose of 400 mg/day (provided tolerability was satisfactory).

The dose was up-titrated only if tolerability of the previous dose level was satisfactory. In the event that subjects experienced adverse events such that, in the investigators' judgment, the dose of lacosamide should not be up-titrated, subjects were permitted to either remain at their current dose level or back-titrate to their previous dose level. Only one back-titration was permitted during the trial. Once the dose of trial medication had been reduced, it could not be re-escalated.

Once subjects had completed the Titration Phase (i.e., after 5 weeks), subjects entered the 4-week Maintenance Phase.

If subjects reached total or sufficient pain relief with lower doses than 400 mg/day, after careful consideration by the investigator, they were allowed to stay on the attained dose level in the Maintenance Phase. If adverse events were intolerable during the Titration Phase or the first week of the Maintenance Phase (only for those subjects reaching the 400 mg/day level), subjects could be down-titrated, once, to the next lowest dose. Subjects were treated at 400 mg/day (or highest dose achieved) for 4 weeks in the Maintenance Phase.

At the end of the Maintenance Phase, subjects entered the Taper Phase and were tapered off the active medication or placebo in a blinded manner (over a period of 1 week). Subjects on 400 mg/day decreased their trial medication by 200 mg during the Taper Phase. Subjects on 300 mg/day reduced their dose of trial medication by 100 mg during the Taper Phase. Subjects on 100 mg/day and 200 mg/day received placebo during the Taper Phase.

### Reference Therapy, Dose and Mode of Administration

Placebo was provided in matching tablets.

### Criteria for Evaluation

### Efficacy

The primary variable was the within-subject change in average daily pain score from the Baseline week to Maintenance Phase, using an 11-point Likert scale (0-10).

Secondary variables included the following:
within-subject change in average daily pain score from the Baseline week to the third week of titration;
within-subject change in average daily pain score from the Baseline week to each week of the trial;
within-subject change in average daily Present Pain Intensity from the Baseline week to each week of the trial;
change in subject's perception of different neuropathic pain qualities (Neuropathic Pain Scale (NPS)) assessed at Visits 1, 3, 6, 10, 11, and 12;
change in subject's perception of pain interference with sleep and activity from the Baseline week to each week of the trial—sleep was assessed every morning (Likert—out of Brief Pain Inventory (BPI)), and activity was assessed every evening (Likert—out of BPI);
change in subject's perception of pain assessed at every clinic visit, as measured by Short Form-McGill Pain Questionnaire (SF-MPQ) in 3 sections:
   categorical pain rating scale, from 0 (no pain) to 3 (severe pain);
   Visual Analog Scale (VAS), rating pain on 100-mm scale;
   Present Pain Intensity (PPI), rating pain on a 6-point scale;
patient's global impression of change in pain (PGIC) completed at Visits 6, 10, and 12;
clinical global impression of change in pain (CGIC) completed at Visits 6, 10, and 12;
change in Short Form-36 (SF-36) Quality of Life (QOL) questionnaire completed at Visits 1, 3, 6, 10, and 12;
change in Profile of Mood States (POMS) questionnaire completed at Visits 1, 3, 6, 10, and 12;
proportion of pain-free days during Baseline, Titration Phase, Maintenance Phase, and Taper Phase;
use (number) of rescue medication.

### Pharmacokinetics

Plasma concentrations of unchanged lacosamide pre-dose (trough) and peak (i.e., 2-4 hours post-dose) concentrations were evaluated.

### Safety

Safety variables evaluated included adverse events, clinical laboratory assessments, electrocardiograms (ECGs), vital sign measurements, and physical and neurological examinations.

### Statistical Methods

It was determined that 46 evaluable subjects in each treatment group would yield an 80% probability of detecting a significant difference at a two-sided 5% level of significance if the true treatment effect was 1.25 units on the Likert scale with a common standard deviation of 2.11.

A total of 70 subjects in each treatment group were to be accrued to allow for subjects who dropped out during the 4-week Run-In Phase or could not be evaluated because of missing Baseline or follow-up observations. It was estimated that 140 subjects would be required to enroll in order to achieve a total of 120 randomized subjects and 100 evaluable subjects for the primary analysis. If the dropout rate was lower than expected, the enrollment was to be stopped before a total of 70 subjects per treatment group were accrued.

For the primary efficacy variable, an analysis of covariance (ANCOVA) with terms for treatment and investigator type was used to compare the difference between active treatment and placebo using the Baseline Likert pain score as a covariate and the change from average Baseline to average Maintenance Phase Likert score as the response. The treatment difference was estimated on the basis of least squares mean (LSMean). A two-sided 95% confidence interval (CI) for the treatment difference was calculated. As a secondary analysis, treatment-by-investigator interaction and/or other potential factors (age, race, sex, and Baseline severity) were explored in the ANCOVA model.

The main effect ANCOVA model was applied to the change in average daily Likert pain score from Baseline to the first three weeks of the Titration Phase, to the entire Titration Phase, to the Treatment Phase, and to each visit using the Baseline value as a covariate.

### Results

### Efficacy Results

Efficacy results from this study consistently demonstrated a statistically significant difference between lacosamide and placebo in subjects with painful diabetic neuropathy with regard to the primary efficacy endpoints tested in this trial. The reduction in mean pain scores following the administration of lacosamide can be regarded as clinically meaningful. Analysis of the secondary efficacy endpoints provided additional statistically significant and clinically relevant results (e.g., clinically meaningful improvements in subjects' quality of sleep and daily routine activity levels).

The primary efficacy variable for this trial was the within-subject change in the average daily pain score from the Baseline week to the Maintenance Phase using an 11-point Likert scale (0-10), where Baseline was the 7-day period between Visits 2 and 3. In the Full Analysis Set (Last Observation Carried Forward, LOCF) there was a 3.11 point reduction in pain from Baseline to the Maintenance Phase following lacosamide treatment compared with a 2.21 point reduction in pain following placebo treatment based on the LSMean. The difference in LSMean pain score between the two groups (0.9) was statistically significant (p=0.0390) and clinically meaningful. Analysis of the FAS (As Observed) and PPS populations for the change from Baseline to the Maintenance Phase also demonstrated greater reductions in pain following treatment with lacosamide than with placebo; these differences were statistically significant and clinically meaningful.

For the FAS (LOCF) and, to an even greater extent the FAS (As Observed), lacosamide was more effective in reducing pain by visit than placebo. By the end of the first 3 weeks of Titration, lacosamide-treated subjects had lower average daily pain scores than placebo-treated subjects. As the dose of lacosamide was escalated through the remainder of the Titration Phase and eventually stabilized during the Maintenance Phase, average daily pain scores were increasingly lower relative to placebo-treated subjects. Tapering lacosamide was associated with subsequent increases in average daily pain score.

Changes in the secondary efficacy endpoints were consistent with those seen in the primary endpoint and provide further support for the efficacy of lacosamide in painful diabetic neuropathy. Statistically significant differences from Baseline to the Maintenance Phase following lacosamide treatment versus placebo were seen for the VAS (rating of overall pain) and present pain intensity of the SF-MPQ, subject's perception of pain interference with sleep, and subject's perception of pain interference with general activity. Statistically significant differences were also observed following lacosamide and placebo treatment for CGIC and PGIC scores at the end of Maintenance Phase (Visit 10). A larger proportion of subjects in the lacosamide group compared with the placebo group experienced a decrease of 2 or more points on the Likert pain scale during all phases of the trial. In addition, treatment with lacosamide was associated with greater improvements than placebo in other pain indices (present pain intensity and subject's perception of different neuropathic pain qualities), quality of life data (SF-36 and POMS), and proportion of pain-free days.

### Pharmacokinetics Results

Trough and peak plasma drug concentrations during the Maintenance Phase showed a wide range of values, explained in part by the variability in subject weight and the low number of samples collected. At the 400 mg/day dose, the mean plasma drug concentrations increased from 7.7 μg/ml to 11.4 μg/ml (trough to peak, Visit 9) and from 7.9 μg/ml to 9.1 μg/ml (Visit 10).

### Safety Results

The mean duration of exposure to study medication was 62.7 days for subjects in the placebo treatment group and 59.6 days for subjects in the lacosamide treatment group indicating high tolerability to lacosamide.

The percentage of subjects who reported at least one treatment-emergent adverse event (TEAE) was higher in the lacosamide treatment group, with overall incidence rates of 87% of 60 subjects in the lacosamide group and 75% of 59 subjects in the placebo group.

Among all subjects, TEAEs were most common in the central and peripheral nervous system, with 46 subjects (39% of 119 subjects) reporting at least one adverse event in this body system. However, adverse events associated with the central and peripheral nervous system were reported by comparable percentages of subjects in each treatment group (39% of 59 placebo subjects, 38% of 60 lacosamide subjects). Review of the adverse event profile provided no evidence for an adverse effect on any particular body system.

Overall, headache (20% of 119 subjects), dizziness (12%), accident not otherwise specified (NOS) (11%), upper respiratory tract infection (10%), and nausea (9%) were the most frequently reported TEAEs. In general, the proportions of subjects who reported specific TEAEs, or who reported TEAEs associated with particular body systems, were comparable between the placebo and lacosamide treatment groups.

Overall, more subjects first reported AEs during the Titration Phase compared with the Maintenance Phase and Taper/Safety Follow-Up Phase. For most body systems, the number of subjects reporting AEs within each body system was greater during the Titration Phase compared with the Maintenance Phase and Taper/Safety Follow-Up Phase.

No subjects died during this study. A total of 2 subjects reported 2 serious adverse events during this study: one subject with one serious adverse event (SAE) in the placebo treatment group (pain in right hip judged to be not related to study medication) and one subject with one SAE in the lacosamide treatment group (abnormal electrocardiogram (ECG) judged to be unlikely related to study medication). A total of 8 subjects (7% of 119 subjects) withdrew from the trial due to an AE: 3 in the placebo group (5% of 59 subjects) and 5 in the lacosamide group (8% of 60 subjects).

Mean and median changes from baseline in hematology, clinical chemistry and coagulation, urinalysis, vital signs, and ECG parameters were small, within normal ranges, comparable between treatment groups, and not of clinical concern. No safety issues with regards to QT or PR interval were identified during the study. HbA_{1C} levels changed only slightly from Baseline to Visit 8 for the two treatment groups (mean changes of −0.1% and 0.1% for the placebo and lacosamide groups, respectively). No important differences were observed in these parameters in the transitions from Baseline to on-therapy values between the two treatment groups. Neurological examination findings at the end of the trial compared with those at Baseline did not suggest any significant effects due to lacosamide.

Body weight changes during treatment with lacosamide were small (−0.5 kg for lacosamide and 1.2 kg for placebo).

### Conclusions

In summary, lacosamide showed statistically significant, clinically meaningful efficacy in reducing neuropathic pain due to diabetic distal sensory polyneuropathy when titrated to a maintenance dose of 400 mg/day. Overall, 60 subjects with painful diabetic neuropathy were treated with lacosamide 100-400 mg/day for up to 82 days; 46 of these subjects completed all phases of the trial. Analyses of safety data (adverse events, clinical laboratory evaluations, ECGs, vital signs, and physical examinations) revealed no serious safety issues and support the further clinical development of lacosamide as an agent to treat diabetic patients with peripheral neuropathic pain.

Lacosamide did not induce weight gain which is an important property for drugs administered to diabetic Type II patients. Some antidiabetic agents such as insulin and sulfonylureas are associated with weight gain (UKPDS 1998) and obesity is an established risk factor for cardiovascular disease (Schernthaner 1996).

### Example 2

This example describes a study demonstrating effectiveness of lacosamide alone and in combination with gabapentin in the rat formalin paw test (late phase), as described by Wheeler-Aceto & Cowan (1991) Psychopharmacology 104:35-44, which detects analgesic activity.

### Materials and Methods

Rats were given an intraplantar injection of 5% formalin (50 μl) into the posterior left paw. This treatment induces a recognizable flinching and licking response of the affected paw in control animals. The number of flinches was counted for 15 minutes, beginning 20 minutes after injection of formalin. The time spent licking the affected paw was also recorded.

Male Rj: Wistar (Han) rats, 10 per group, weighing 100-130 g at the beginning of the experiments were studied per group. The test was performed blind.

Lacosamide (20 mg/kg), gabapentin (50 and 100 mg/kg), combinations of lacosamide (20 mg/kg) with gabapentin (50 and 100 mg/kg), and vehicle were administered i.p. 10 minutes before injection of formalin.

### Results

Results of the test are presented in Tables 1 (number of flinches) and 2 (licking time).

TABLE 1
Effect of lacosamide, gabapentin and combinations on number of flinches

| Compound 1 | Compound 2 | No. of flinches | | | |
|---|---|---|---|---|---|
| (mg/kg) | (mg/kg) | mean ± SEM | | p value | % change |
| Vehicle | Vehicle | 127.8 ± 21.2 | | — | — |
| Lacosamide (20) | Vehicle | 85.7 ± 14.3 | NS (a) | 0.1736 | −33% (a) |
| Vehicle | Gabapentin (50) | 97.4 ± 23.8 | NS (a) | 0.3445 | −24% (a) |
| Vehicle | Gabapentin (100) | 88.1 ± 19.4 | NS (a) | 0.2121 | −31% (a) |
| Lacosamide (20) | Gabapentin (50) # | 46.0 ± 21.1 | ** (a) | 0.0071 | −64% (a) |
| | | | * (b) | 0.0222 | −46% (b) |
| | | | NS (c) | 0.0790 | −53% (c) |
| Lacosamide (20) | Gabapentin (100) | 31.0 ± 9.3 | ** (a) | 0.0017 | −76% (a) |
| | | | ** (b) | 0.0041 | −64% (b) |
| | | | * (c) | 0.0343 | −65% (c) |

TABLE 2
Effect of lacosamide, gabapentin and combinations on licking time

| Compound 1 | Compound 2 | Licking time (seconds) | | | |
|---|---|---|---|---|---|
| (mg/kg) | (mg/kg) | mean ± SEM | | p value | % change |
| Vehicle | Vehicle | 222.4 ± 33.8 | | — | — |
| Lacosamide (20) | Vehicle | 146.9 ± 23.8 | NS (a) | 0.0962 | −34% (a) |
| Vehicle | Gabapentin (50) | 161.0 ± 27.3 | NS (a) | 0.2258 | −28% (a) |
| Vehicle | Gabapentin (100) | 90.0 ± 22.5 | * (a) | 0.0101 | −60% (a) |
| Lacosamide (20) | Gabapentin (50) # | 58.6 ± 32.0 | ** (a) | 0.0042 | −74% (a) |
| | | | * (b) | 0.0220 | −60% (b) |
| | | | * (c) | 0.0365 | −64% (c) |
| Lacosamide (20) | Gabapentin (100) | 39.1 ± 19.9 | *** (a) | 0.0007 | −82% (a) |
| | | | ** (b) | 0.0022 | −73% (b) |
| | | | NS (c) | 0.0685 | −57% (c) |

NS = not significant;
* = p < 0.05;
** = p < 0.01;
*** = p < 0.001
(a): compared with vehicle control
(b): compared with lacosamide alone at the appropriate dose
(c): compared with gabapentin alone at the appropriate dose
#: missing value (1/10)

Lacosamide alone at 20 mg/kg tended to decrease the number of flinches by 33% as compared with vehicle controls. It also tended to decrease the time spent licking, by 34% as compared with vehicle controls (p=0.0962).

Gabapentin alone at 50 and 100 mg/kg globally but non-significantly decreased the number of flinches, by 24% and 31% respectively as compared with vehicle controls. Gabapentin dose-dependently decreased the time spent licking, by 28% (50 mg/kg) and 60% (100 mg/kg), significantly so at 100 mg/kg (p<0.05).

Lacosamide 20 mg/kg combined with gabapentin 50 and 100 mg/kg clearly and dose-dependently decreased the number of flinches, by 64% and 76% respectively (p<0.01) as compared with vehicle controls. The combination clearly and dose-dependently decreased the time spent licking, by 74% (p<0.01) and 82% (p<0.001) respectively. The effects of lacosamide combined with gabapentin on the number of flinches and the time spent licking were significantly more marked than the effects of lacosamide alone (p<0.05 or p<0.01).

### Example 3

This example describes a study demonstrating effectiveness of lacosamide alone and in combination with morphine in the rat formalin paw test (late phase), as described by Wheeler-Aceto & Cowan (1991), supra.

### Materials and Methods

Test methods were similar to those of Example 2. Lacosamide (10 and 20 mg/kg), morphine (2 and 4 mg/kg), combinations of lacosamide (10 and 20 mg/kg) with morphine (2 and 4 mg/kg), and vehicle were administered i.p. 10 minutes before injection of formalin.

### Results

Results of the test are presented in Tables 3 (number of flinches) and 4 (licking time).

TABLE 3
Effect of lacosamide, morphine and combinations on number of flinches

| Compound 1 | Compound 2 | No. of flinches | | | |
|---|---|---|---|---|---|
| (mg/kg) | (mg/kg) | mean ± SEM | | p value | % change |
| Vehicle | Vehicle | 150.0 ± 21.0 | | — | — |
| Lacosamide (10) | Vehicle | 182.7 ± 25.9 | NS (a) | 0.3254 | +22% (a) |
| Lacosamide (20) | Vehicle | 97.2 ± 16.0 | NS (a) | 0.0961 | −35% (a) |
| Vehicle | Morphine (2) | 139.5 ± 25.3 | NS (a) | 0.6499 | −7% (a) |
| Vehicle | Morphine (4) | 94.3 ± 21.1 | NS (a) | 0.1303 | −37% (a) |
| Lacosamide (10) | Morphine (2) | 139.7 ± 29.4 | NS (a) | 0.7621 | −7% (a) |
| | | | NS (b) | 0.3638 | −24% (b) |
| | | | NS (c) | 0.8205 | 0% (c) |
| Lacosamide (10) | Morphine (4) | 20.6 ± 7.9 | *** (a) | 0.0002 | −86% (a) |
| | | | *** (b) | 0.0003 | −89% (b) |
| | | | ** (c) | 0.0035 | −78% (c) |
| Lacosamide (20) | Morphine (2) | 44.7 ± 12.3 | ** (a) | 0.0015 | −70% (a) |
| | | | * (b) | 0.0342 | −54% (b) |
| | | | ** (c) | 0.0091 | −68% (c) |
| Lacosamide (20) | Morphine (4) | 19.6 ± 13.3 | *** (a) | 0.0005 | −87% (a) |
| | | | ** (b) | 0.0014 | −80% (b) |
| | | | ** (c) | 0.0024 | −79% (c) |

TABLE 4
Effect of lacosamide, morphine and combinations on licking time

| Compound 1 | Compound 2 | Licking time (seconds) | | | |
|---|---|---|---|---|---|
| (mg/kg) | (mg/kg) | mean ± SEM | | p value | % change |
| Vehicle | Vehicle | 291.9 ± 25.6 | | — | — |
| Lacosamide (10) | Vehicle | 210.1 ± 22.7 | * (a) | 0.0191 | −28% (a) |
| Lacosamide (20) | Vehicle | 128.2 ± 28.0 | *** (a) | 0.0009 | −56% (a) |
| Vehicle | Morphine (2) | 289.3 ± 30.7 | NS (a) | 0.7054 | −1% (a) |
| Vehicle | Morphine (4) | 234.9 ± 37.3 | NS (a) | 0.4055 | −20% (a) |
| Lacosamide (10) | Morphine (2) | 212.1 ± 27.2 | NS (a) | 0.1304 | −27% (a) |
| | | | NS (b) | 0.7624 | +1% (b) |
| | | | * (c) | 0.0284 | −27% (c) |
| Lacosamide (10) | Morphine (4) | 150.9 ± 36.3 | ** (a) | 0.0051 | −48% (a) |
| | | | NS (b) | 0.2265 | −28% (b) |
| | | | NS (c) | 0.1306 | −36% (c) |
| Lacosamide (20) | Morphine (2) | 91.5 ± 25.7 | *** (a) | 0.0004 | −69% (a) |
| | | | NS (b) | 0.2258 | −29% (b) |
| | | | *** (c) | 0.0009 | −68% (c) |
| Lacosamide (20) | Morphine (4) | 17.1 ± 16.4 | *** (a) | 0.0001 | −94% (a) |
| | | | ** (b) | 0.0018 | −87% (b) |
| | | | *** (c) | 0.0003 | −93% (c) |

NS = not significant;
* = p < 0.05;
** = p < 0.01;
*** = p < 0.001
(a): compared with vehicle control
(b): compared with lacosamide alone at the appropriate dose
(c): compared with morphine alone at the appropriate dose

Lacosamide alone at 10 and 20 mg/kg did not strongly affect the number of flinches, as compared with vehicle controls (22% increase and 35% decrease, respectively) although the tendency towards a decrease at 20 mg/kg approached statistical significance (p=0.0961). Lacosamide dose-dependently decreased the time spent licking by 28% (p<0.05) at 10 mg/kg and by 56% (p<0.001) at 20 mg/kg.

Morphine alone at 2 and 4 mg/kg dose-dependently decreased the number of flinches and the time spent licking, as compared with vehicle controls. Nevertheless, these effects did not reach statistical significance.

Lacosamide 10 mg/kg combined with morphine 4 mg/kg, but not with morphine 2 mg/kg, clearly decreased the number of flinches by 86% (p<0.001) and the time spent licking by 48% (p<0.01), as compared with vehicle controls. The effects of lacosamide 10 mg/kg combined with morphine 4 mg/kg on the number of flinches, but not on the time spent licking, were more marked than the effects of lacosamide alone at the same dose (p<0.001).

Lacosamide 20 mg/kg combined with morphine 2 and 4 mg/kg clearly and dose-dependently decreased the number of flinches by 70% (p<0.01) and 87% (p<0.001) respectively, as compared with vehicle controls. The combination clearly and dose-dependently decreased the time spent licking by 69% and 94%, respectively (p<0.001). The effects of lacosamide 20 mg/kg combined with morphine on the number of flinches and the time spent licking were significantly more marked than the effects of lacosamide alone at the same dose (p<0.05 or p<0.01), except for the time spent licking at the 2 mg/kg dose of morphine.

### Example 4

This example describes a study demonstrating effectiveness of lacosamide alone and in combination with the antidepressant duloxetine in the rat formalin paw test (late phase), as described by Wheeler-Aceto & Cowan (1991), supra.

### Materials and Methods

Test methods were similar to those of Example 2. Lacosamide (10 mg/kg), duloxetine (8 mg/kg), a combination of lacosamide (10 mg/kg) with duloxetine (8 mg/kg), and vehicle were administered i.p. 10 minutes before injection of formalin.

### Results

This example describes a study demonstrating effectiveness of lacosamide alone and in combination with the antidepressant duloxetine in the rat formalin paw test (late phase), as described by Wheeler-Aceto & Cowan (1991), supra.

### Materials and Methods

Test methods were similar to those of Example 2. Lacosamide (10 mg/kg), duloxetine (8 mg/kg), a combination of lacosamide (10 mg/kg) with duloxetine (8 mg/kg), and vehicle were administered i.p. 10 minutes before injection of formalin.

### Results

Results of the test are presented in Tables 5 (number of flinches) and 6 (licking time).

TABLE 5
Effect of lacosamide, duloxetine and combination on number of flinches

| Compound 1 | Compound 2 | No. of flinches | | | |
|---|---|---|---|---|---|
| (mg/kg) | (mg/kg) | mean ± SEM | | p value | % change |
| Vehicle | Vehicle | 151.3 ± 13.7 | | — | — |
| Lacosamide (10) | Vehicle | 158.2 ± 15.6 | NS (a) | 0.5963 | +5% (a) |
| Vehicle | Duloxetine (8) | 149.6 ± 27.3 | NS (a) | 0.7054 | −1% (a) |
| Lacosamide (10) | Duloxetine (8) | 105.1 ± 11.3 | * (a) | 0.0233 | −31% (a) |
| | | | * (b) | 0.0284 | −34% (b) |
| | | | NS (c) | 0.1988 | −30% (c) |

TABLE 6
Effect of lacosamide, duloxetine and combination on licking time

| Compound 1 | Compound 2 | Licking time (seconds) | | | |
|---|---|---|---|---|---|
| (mg/kg) | (mg/kg) | mean ± SEM | | p value | % change |
| Vehicle | Vehicle | 264.2 ± 17.8 | | — | — |
| Lacosamide (10) | Vehicle | 185.2 ± 31.7 | NS (a) | 0.0538 | −30% (a) |
| Vehicle | Duloxetine (8) | 195.5 ± 45.0 | NS (a) | 0.1615 | −26% (a) |
| Lacosamide (10) | Duloxetine (8) | 96.9 ± 24.8 | *** (a) | 0.0004 | −63% (a) |
| | | | * (b) | 0.0340 | −48% (b) |
| | | | NS (c) | 0.1492 | −50% (c) |

NS = not significant;
* = p < 0.05;
** = p < 0.01;
*** = p < 0.001
(a): compared with vehicle control
(b): compared with lacosamide alone at the appropriate dose
(c): compared with duloxetine alone at the appropriate dose

Lacosamide 10 mg/kg alone had no significant effects although it tended to decrease the time spent licking (30% decrease, p=0.0538).

Duloxetine 8 mg/kg alone had no clear effects.

Lacosamide 10 mg/kg combined with duloxetine 8 mg/kg significantly decreased the number of flinches, as compared with vehicle controls, by 31% (p<0.05). The combination decreased the time spent licking by 63% (p<0.001). The effects of lacosamide combined with duloxetine on the number of flinches and the time spent licking were more marked than the effects of lacosamide alone (p<0.05 to p<0.01).

### Example 5

This example describes a study demonstrating effectiveness of lacosamide alone and in combination with the NMDA receptor antagonist memantine in the rat formalin paw test (late phase), as described by Wheeler-Aceto & Cowan (1991), supra.

### Materials and Methods

Test methods were similar to those of Example 2. Lacosamide (10 and 20 mg/kg), memantine (4 and 8 mg/kg), combinations of lacosamide (10 and 20 mg/kg) with memantine (4 and 8 mg/kg), and vehicle were administered i.p. 10 minutes before injection of formalin.

### Results

Results of the test are presented in Tables 7 (number of flinches) and 8 (licking time).

TABLE 7
Effect of lacosamide, memantine and combinations on number of flinches

| Compound 1 | Compound 2 | No. of flinches | | | |
|---|---|---|---|---|---|
| (mg/kg) | (mg/kg) | mean ± SEM | | p value | % change |
| Vehicle | Vehicle | 165.6 ± 20.1 | | — | — |
| Lacosamide (10) | Vehicle | 113.9 ± 23.2 | NS (a) | 0.0821 | −31% (a) |
| Lacosamide (20) | Vehicle | 85.8 ± 14.4 | * (a) | 0.0101 | −48% (a) |
| Vehicle | Memantine (4) | 161.4 ± 26.3 | NS (a) | 0.7052 | −3% (a) |
| Vehicle | Memantine (8) | 132.3 ± 24.6 | NS (a) | 0.3845 | −20% (a) |
| Lacosamide (10) | Memantine (4) | 105.4 ± 16.1 | * (a) | 0.0211 | −36% (a) |
| | | | NS (b) | 0.8205 | −7% (b) |
| | | | NS (c) | 0.1124 | −35% (c) |
| Lacosamide (10) | Memantine (8) | 83.5 ± 23.4 | * (a) | 0.0311 | −50% (a) |
| | | | NS (b) | 0.2568 | −27% (b) |
| | | | NS (c) | 0.1988 | −37% (c) |
| Lacosamide (20) | Memantine (4) | 42.5 ± 9.0 | *** (a) | 0.0004 | −74% (a) |
| | | | * (b) | 0.0257 | −50% (b) |
| | | | *** (c) | 0.0004 | −74% (c) |
| Lacosamide (20) | Memantine (8) | 59.6 ± 11.0 | *** (a) | 0.0007 | −64% (a) |
| | | | NS (b) | 0.1986 | −31% (b) |
| | | | * (c) | 0.0283 | −55% (c) |

TABLE 8
Effect of lacosamide, memantine and combinations on licking time

| Compound 1 | Compound 2 | Licking time (seconds) | | | |
|---|---|---|---|---|---|
| (mg/kg) | (mg/kg) | mean ± SEM | | p value | % change |
| Vehicle | Vehicle | 176.3 ± 18.2 | | — | — |
| Lacosamide (10) | Vehicle | 168.5 ± 23.9 | NS (a) | 0.8797 | −4% (a) |
| Lacosamide (20) | Vehicle | 85.1 ± 19.1 | ** (a) | 0.0072 | −52% (a) |
| Vehicle | Memantine (4) | 219.9 ± 21.8 | NS (a) | 0.0537 | +25% (a) |
| Vehicle | Memantine (8) | 237.3 ± 18.9 | * (a) | 0.0412 | +35% (a) |
| Lacosamide (10) | Memantine (4) # | 168.2 ± 26.1 | NS (a) | 0.7749 | −5% (a) |
| | | | NS (b) | 0.9349 | 0% (b) |
| | | | NS (c) | 0.1208 | −24% (c) |
| Lacosamide (10) | Memantine (8) | 114.8 ± 18.8 | * (a) | 0.0342 | −35% (a) |
| | | | NS (b) | 0.1508 | −32% (b) |
| | | | ** (c) | 0.0015 | −52% (c) |
| Lacosamide (20) | Memantine (4) | 54.1 ± 10.5 | *** (a) | 0.0002 | −69% (a) |
| | | | NS (b) | 0.3071 | −36% (b) |
| | | | *** (c) | 0.0007 | −75% (c) |
| Lacosamide (20) | Memantine (8) | 90.6 ± 26.8 | * (a) | 0.0191 | −49% (a) |
| | | | NS (b) | 0.8500 | +6% (b) |
| | | | ** (c) | 0.0015 | −62% (c) |

NS = not significant;
* = p < 0.05;
** = p < 0.01;
*** = p < 0.001
(a): compared with vehicle control
(b): compared with lacosamide alone at the appropriate dose
(c): compared with memantine alone at the appropriate dose
#: missing value (1/10)

Lacosamide alone at 10 and 20 mg/kg dose-dependently decreased the number of flinches, as compared with vehicle controls, by 31% and 48% respectively, significantly so at 20 mg/kg (p<0.05). Lacosamide clearly decreased the time spent licking at 20 mg/kg (52% decrease, p<0.01) but had no clear effects at 10 mg/kg.

Memantine alone at 4 and 8 mg/kg did not clearly affect the number of flinches, as compared with vehicle controls. Memantine dose-dependently increased the time spent licking (25% increase, p=0.0537 and 35% increase, p<0.05).

Lacosamide at 10 mg/kg combined with memantine at 4 and 8 mg/kg dose-dependently decreased the number of flinches, as compared with vehicle controls, by 36% and 50% respectively (p<0.05). The combination significantly decreased the time spent licking at 8 but not at 4 mg/kg of memantine (35% decrease, p<0.05). The effects of lacosamide combined with memantine on the number of flinches and the time spent licking were not different from the effects of lacosamide alone.

Lacosamide at 20 mg/kg combined with memantine at 4 and 8 mg/kg clearly decreased the number of flinches, as compared with vehicle controls, by 74% and 64% respectively (p<0.001). The combination clearly decreased the time spent licking, although in a manner inversely related to the dose of memantine (69% decrease, p<0.001 and 49% decrease, p<0.05, respectively). The effects of lacosamide combined with memantine at 4 mg/kg on the number of flinches but not on the time spent licking were significantly more marked than the effects of lacosamide alone (p<0.05).

All patents and publications cited herein are incorporated by reference into this application in their entirety.

The words “comprise”, “comprises”, and “comprising” are to be interpreted inclusively rather than exclusively.

1. A method for treating pain in painful diabetic neuropathy in a subject comprising administering in combination to the subject a first agent comprising a compound of
Formula (III) 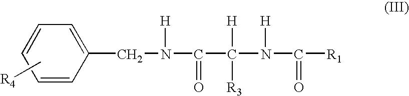
wherein:
R₄ is one or more substituents independently selected from the group consisting of hydrogen and halo;
R₃ is selected from the group consisting methoxymethyl, phenyl, N-methoxy-N-methylamino, and N-methoxyamino; and
R₁ is lower alkyl
or a pharmaceutically acceptable salt thereof; and a second agent effective in combination therewith to provide enhanced treatment of pain, by comparison with the first agent alone, wherein the second agent is selected from the group consisting of gabapentin, morphine, duloxetine, memantine, and pregabalin.

2. (canceled)

3. (canceled)

4. (canceled)

5. The method of claim 1, wherein, in the compound of Formula (III), R₃ is methoxymethyl.

6. The method of claim 1, wherein, in the compound of Formula (III),
no more than one R₄ substituent is fluoro and all others are hydrogen;
and
R₁ is methyl.

7. The method of claim 1, wherein, in the compound of Formula (III),
R₄ is hydrogen;
R₃ is methoxymethyl; and
R₁ is methyl.

8. The method of claim 1, wherein the compound of Formula (III) is selected from the group consisting of
(R)-2-acetamido-N-benzyl-3-methoxy-propionamide;
(R)-2-acetamido-N-benzyl-3-ethoxy-propionamide;
O-methyl-N-acetyl-D-serine-m-fluorobenzylamide;
O-methyl-N-acetyl-D-serine-p-fluorobenzylamide; and.

9. The method of claim 1, wherein the compound of Formula (III) is substantially enantiopure.

10. The method of claim 1, wherein the compound of Formula (III) is lacosamide.

11. (canceled)

12. The method of claim 10, wherein the lacosamide is administered at a dose of about 100 to about 1000 mg/day.

13. The method of claim 10, wherein the lacosamide is administered at a dose of about 200 to about 600 mg/day.

14. The method of claim 10, wherein the lacosamide is administered in an amount providing a daily dose effective to provide a plasma concentration of lacosamide of about 0.1 to about 15 μg/ml (trough) and about 5 to about 18.5 μg/ml (peak), calculated as an average over a plurality of treated subjects.

15. The method of claim 1, wherein the compound of Formula (III) is administered according to a regimen wherein daily doses are increased until a predetermined daily dose is reached which is maintained during further treatment.

16. The method of claim 1, wherein the compound of Formula (III) is administered in one to three doses per day.

17. The method of claim 1, wherein the compound of Formula (III) is administered orally or intravenously.

18. (canceled)

19. (canceled)

20. (canceled)

21. (canceled)

22. (canceled)

23. The method of claim 1, wherein the painful diabetic neuropathy is diabetic distal sensory polyneuropathy.

24. The method of claim 1, wherein the first agent and second agent are, in combination, effective for treatment of an aspect of pain selected from the group consisting of average daily pain, overall pain, present pain intensity, pain interference with sleep, the subject's perception of pain interference with general activity, the subject's global impression of change in pain, clinical global impression of change in pain, the subject's perception of different neuropathic pain qualities, quality of life and proportion of pain-free days.

25. The method of claim 1, wherein the painful diabetic neuropathy is associated with diabetes mellitus Type I.

26. The method of claim 1, wherein the first agent and second agent are administered in separate dosage forms by the same or different routes at the same or different times.

27. The method of claim 1, wherein the first agent and second agent are administered together in a single pharmaceutical dosage form further comprising at least one pharmaceutically acceptable excipient.

28. The method of claim 1, wherein the enhanced treatment of pain comprises greater reduction of intensity and/or duration of pain by comparison with the first agent alone.

29. The method of claim 10, wherein lacosamide is administered in an oral dosage amount of about 50 mg/day to about 600 mg/day.

30. The method of claim 10, wherein lacosamide is administered in an oral dosage amount of about 100 mg/day to about 400 mg/day.

31. A method for treating pain in painful diabetic neuropathy in a subject comprising administering in combination to the subject lacosamide in an oral dosage amount of about 50 mg/day to about 600 mg/day, and a second agent effective in combination with lacosamide to provide enhanced treatment of pain, by comparison with lacosamide alone, wherein the second agent is selected from the group consisting of gabapentin, morphine, duloxetine, memantine, and pregabalin.

32. The method of claim 1, wherein the painful diabetic neuropathy is associated with diabetes mellitus Type II.

33. The method of claim 10 or 31, wherein the second agent is gabapentin.

34. The method of claim 10 or 31, wherein the second agent is pregabalin.
